(19) United States
(12) Patent Application Publication
(10) Pub. No.: US 2015/0177103 A1
(43) Pub. Date: Jun. 25, 2015

(54) APPARATUS AND METHOD FOR DETERMINATION OF CYLINDER HEAD GASKET JOINT FAILURE IN A RUNNING ENGINE

(71) Applicants: Cecil L. Brown, Auburn Hills, MI (US); Michael Jurj, Macomb, MI (US); Ronald W. Berndt, Bloomfield Hills, MI (US); Richard D. Michell, Farmington Hills, MI (US)

(72) Inventors: Cecil L. Brown, Auburn Hills, MI (US); Michael Jurj, Macomb, MI (US); Ronald W. Berndt, Bloomfield Hills, MI (US); Richard D. Michell, Farmington Hills, MI (US)

(21) Appl. No.: 14/135,926

(22) Filed: Dec. 20, 2013

(51) Int. Cl. G01M 15/10; G01N 33/00

(52) U.S. Cl. CPC G01M 15/102; G01N 33/0006

(57) ABSTRACT

An apparatus for determining the existence of a head-gasket failure in an engine is provided. The apparatus includes an accumulator in fluid communication with the engine, an engine coolant flow path in fluid communication with the engine and with the accumulator, and a gas flow path fluidly coupled to the accumulator. The apparatus further includes at least one gas analyzer fluidly coupled to the accumulator via the gas flow path that receives a sample gas from the accumulator via the gas flow path to allow the at least one gas analyzer to detect an amount of carbon dioxide in the sample gas.

### FIELD

The present disclosure relates to an apparatus and method for determination of a seal failure in a cylinder head gasket, and more particularly to a method and apparatus for determination of a cylinder head-gasket failure in a running engine.

### BACKGROUND

This section provides background information related to the present disclosure which is not necessarily prior art.

Head gaskets are used in vehicle engines to maintain a sealed interface between a cylinder head and an engine block of the engine. The head gasket creates a seal retaining the pressure produced by combustion of fuel and air within the engine. In so doing, the head gasket allows the kinetic energy associated with the pressure to be directed downward toward pistons of the engine and, ultimately, through the connecting rod respectively associated with the pistons, thereby causing rotation of a crankshaft. When a head gasket fails, the seal between the cylinder head and the engine block is broken which, in some cases, leads to engine failure, as insufficient pressure is retained and directed toward the pistons.

The failure of a head gasket to maintain a sealed interface between a cylinder head and an engine block of an engine allows byproducts of the engine's combustion process to enter a cooling system associated with the engine. Namely, carbon dioxide is permitted to migrate from combustion chambers of the engine and into the cooling system. Such carbon dioxide typically goes unnoticed until other conditions associated with head-gasket failure are realized (i.e., white smoke coming from the exhaust system, vehicle overheating, engine not running properly, engine failure, etc.).

Conventional systems used to detect head-gasket failure typically require removal of an engine from a test fixture or partial disassembly when in a vehicle. For example, conventional systems typically require removal and partial disassembly of an engine to allow replacement of spark plugs with adapters or test fixtures. Once installed, nitrogen is used to pressurize the engine via the adapters to determine whether the interface between the cylinder head and the engine block is properly sealed by the head gasket. While such methods are effective in determining whether the head gasket adequately seals the interface between the cylinder head and the engine block, such methods are time consuming and costly. Namely, the adapters used to pressurize the engine are typically expensive and, further, are costly to use—given that the engine must be removed from a test fixture and/or partially disassembled before the adapters can be used. Removal of an engine from a test fixture or vehicle obviously results in the test fixture or vehicle being idle and unusable until the engine is reassembled and installed in the test fixture or vehicle.

### SUMMARY

In one configuration, an apparatus for determining the existence of a head-gasket failure in an engine is provided. The apparatus includes an accumulator in fluid communication with the engine, an engine coolant flow path in fluid communication with the engine and with the accumulator, and a gas flow path fluidly coupled to the accumulator. The apparatus further includes at least one gas analyzer fluidly coupled to the accumulator via the gas flow path that receives a sample gas from the accumulator via the gas flow path to allow the at least one gas analyzer to detect an amount of carbon dioxide in the sample gas.

In another configuration, a method for determining the existence of a head-gasket failure in an engine is provided. The method includes providing an engine and an engine cooling system in fluid communication with the engine, providing an accumulator in fluid communication with the engine cooling system and having a quantity of sample gas, and providing a flow path in fluid communication with the accumulator that includes at least one gas analyzer. The method also includes circulating at least a portion of the sample gas through the flow path including the at least one gas analyzer and determining an amount of carbon dioxide in the sample gas.

Further areas of applicability of the teachings of the present disclosure will become apparent from the detailed description, claims and the drawings provided hereinafter, wherein like reference numerals refer to like features throughout the several views of the drawings. It should be understood that the detailed description, including disclosed embodiments and drawings referenced therein, are merely exemplary in nature intended for purposes of illustration only and are not intended to limit the scope of the present disclosure, its application or uses. Thus, variations that do not depart from the gist of the present disclosure are intended to be within the scope of the present disclosure.

### BRIEF DESCRIPTION OF THE DRAWINGS

FIG. 1 is a schematic representation of a system for determining the occurrence of a head-gasket failure, in accordance with the principles of the present disclosure;

FIG. 2 is a flow diagram of a method of calibrating the system of FIG. 1 for determining the occurrence of a head-gasket failure, in accordance with the principles of the present disclosure;

FIG. 3 is a flow diagram of a method of determining a threshold value of carbon dioxide that is indicative of an occurrence of a head-gasket failure, in accordance with the principles of the present disclosure; and

FIG. 4 is a flow diagram of a method for determining the occurrence of a head-gasket failure, in accordance with the principles of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

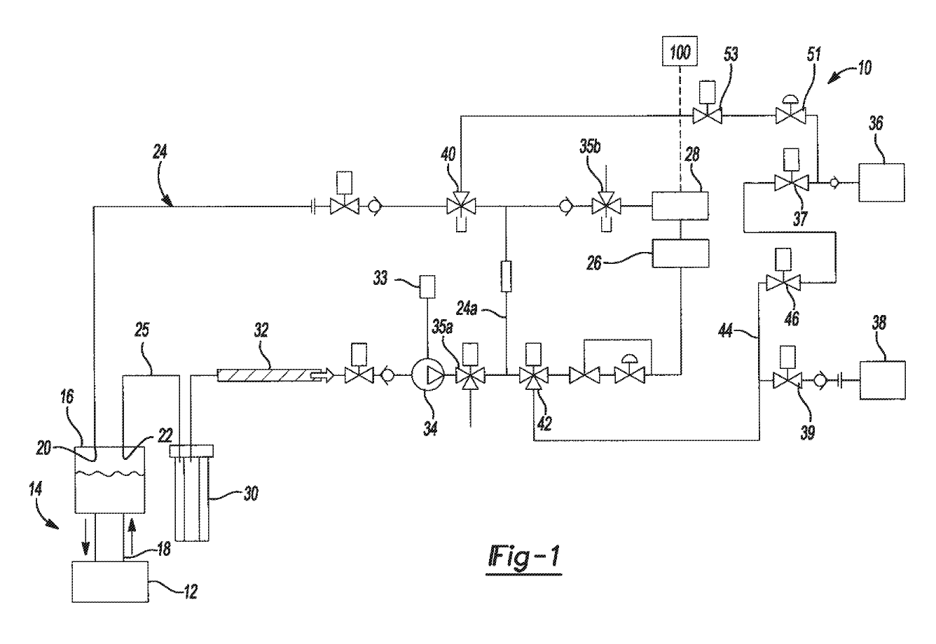

With reference to FIG. 1, a system 10 for detecting the occurrence of a head gasket leak or failure is provided. The system 10 includes an engine 12 and a cooling system 14. The engine 12 includes at least one cylinder (none shown), a cylinder head (not shown) and a head gasket (not shown). The head gasket is designed to seal an interface between the cylinder and the cylinder head, and otherwise seal the cylinder from the cooling system 14. In one configuration, the system 10 detects the occurrence of a failure of the head gasket in providing a sealed interface between the cooling system 14 and the cylinder while the engine 12 is operating.

It will be appreciated that, while the system 10 is generally described herein as detecting the occurrence of a head-gasket failure, the system 10 may also detect the occurrence of any failure in the segregation of, or sealed interface between, the cooling system 14 and the cylinder(s) of the engine 12. In addition, while the system 10 is shown associated with a single engine 12, the system 10 may be coupled to any number of engines 12 to allow the system to detect a failure in the sealed interface between the cooling systems 14 and each of the cylinders of the engines 12.

The cooling system 14 includes a reservoir or accumulator 16 and a coolant flow path 18 under the force of a fluid pump (not shown) to cool the engine 12 during use. The coolant flow path 18 may cool the engine 12 by circulating the coolant from the accumulator 16 through passageways (not shown) formed in the engine 12. The cooling system 14 additionally includes a heat exchanger such as a radiator (not shown) that is in fluid communication with the engine 12 and the accumulator 16 via the coolant flow path 18 to reject heat absorbed from the engine 12 by the coolant.

The accumulator 16 includes an input port 20 and an output port 22. The input port 20 and/or the output port 22 are in fluid communication with a sample fluid flow path 24. References to upstream and downstream location, provided herein, are described from the perspective of the output port 22 being the beginning of the fluid flow path 24, and the input port 20 being the end of the fluid flow path 24.

The fluid flow path 24 includes a sample conditioning device 26 and a fluid analyzer device 28, and circulates a fluid (e.g., air) between the accumulator 16 and the devices 26, 28. The fluid analyzer device 28 detects a contaminant (e.g., carbon dioxide) in the fluid flow path 24 and outputs a value (i.e., an output value) from an output scale that corresponds to a concentration of contaminant in the fluid flow path 24. In one configuration, the sample conditioning device 26 may be the VIA-510 analyzer from HORIBA™, and the fluid analyzer device 28 may be the ES-510 analyzer from HORIBA™.

A filter 30, a heated gas transfer conduit 32, and a pump 34 are located along the fluid flow path 24. The filter 30 controls the quantity of a contaminant (e.g., liquid) in the fluid flow path 24. Namely, the filter 30 removes liquid coolant from a line 25 extending between the outlet port 22 and the filter 30 to prevent liquid from entering the fluid flow path 24. The heated gas transfer conduit 32 controls the temperature of the fluid in the fluid flow path 24 and may include a resistive wire wrapped around a conduit of the fluid flow path 24. As with the filter 30, the heated gas transfer conduit 32 likewise serves to remove condensation from the fluid prior to the fluid reaching the fluid analyzer device 28. The pump 34 creates a circulatory flow of fluid in the fluid flow path 24. A controller 33 is in communication with the pump 34 to control the flow rate of the fluid in the fluid flow path 24. In one configuration, the filter 30 is located downstream of the accumulator 16 and upstream of the heated gas transfer conduit 32 while the pump 34 is located downstream of the heated gas transfer conduit 32 and upstream of the fluid analyzer device 28, as shown in FIG. 1.

A first exhaust valve 35a is located upstream of the fluid analyzer device 28, and a second exhaust valve 35b is located downstream of the fluid analyzer device 28 (FIG. 1). As will be described in more detail below, the first exhaust valve 35a controls the discharge of fluid from the fluid flow path 24 and controls the flow of fluid between a circulation flow path 24a and the fluid analyzer device 28. The second exhaust valve 35b controls the discharge of fluid from the fluid flow path 24.

A first fluid source 36 and a second fluid source 38 are additionally in fluid communication with the fluid flow path 24. In one configuration, the first fluid source 36 is a zero-gas (e.g., clean air) source and the second fluid source is a span-gas source having a known concentration of a substance or contaminant (e.g., carbon dioxide). The first fluid source 36 is in fluid communication with the second fluid source 38 through an auxiliary flow path 44. At least one valve 46 is disposed downstream of the first fluid source 36 and upstream of the second fluid source 38 in the auxiliary flow path 44.

The first fluid source 36 is in fluid communication with the fluid flow path 24 through a first valve 40. The first valve 40 is located downstream of the fluid analyzer device 28 and downstream of the circulation flow path 24a. The second fluid source 38 is in fluid communication with the fluid flow path 24 through a second valve 42. The second valve 42 is located upstream of the fluid analyzer device 28 and downstream of the pump 34. As shown in FIG. 1, the first valve 40 is disposed downstream of the fluid analyzer device 28 and downstream of the second valve 42 while the second valve 42 is disposed upstream of the fluid analyzer device 28.

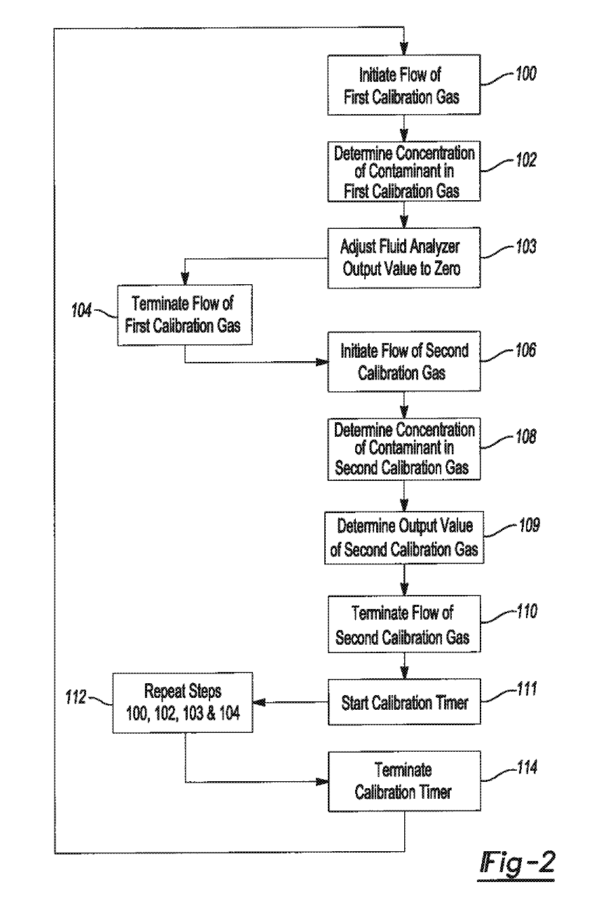

Operation of the system 10 will now be described in detail. With reference to FIGS. 1 and 2, in a first mode of operation, the system 10 calibrates the fluid analyzer device 28 by supplying the fluid analyzer device 28 with span gas having a known concentration of carbon dioxide. During one stage of the first mode of operation, the first fluid source 36 is in fluid communication with the fluid flow path 24 to provide zero gas (i.e., clean gas) to at least a portion of the fluid flow path 24 and the fluid analyzer device 28. Namely, the valves 37 and 46 are opened to cause fluid to flow from the first fluid source 36 and through the auxiliary flow path 44. The second valve 42 is likewise opened to cause fluid to flow from the auxiliary flow path 44, through the fluid flow path 24, and through the fluid analyzer device 28. The second exhaust valve 35b is also opened such that fluid is discharged from the system 10 downstream of the fluid analyzer device 28. In this way, zero gas from the first fluid source 36 operates to remove contaminants and other fluids, such as span gas or a sample gas, from the fluid flow path 24 prior to calibrating the fluid analyzer device 28.

During another stage of the first mode of operation, the second fluid source 38 is placed in fluid communication with the fluid flow path 24 by opening a valve 39 associated with the second fluid source 38 to provide span gas (i.e., gas at a known concentration of carbon dioxide) to at least a portion of the fluid flow path 24 and to the fluid analyzer device 28. During this stage, the second valve 42 is opened such that fluid from the second fluid source 38 flows through the fluid flow path 24 and through the fluid analyzer device 28. In addition, the second exhaust valve 35b is also opened, such that fluid is discharged from the system 10 downstream of the fluid analyzer device 28.

As described, the first fluid source 36 flows through the fluid analyzer device 28 prior to being expelled from the system at the second exhaust valve 35b. At this point, the output value of the fluid analyzer device 28 is adjusted electronically to correspond to a zero value of the concentration of the contaminant in the fluid flow path 24, and the valves 37 and 46 are subsequently closed. The valve 39 associated with the second fluid source 38 is then opened to permit span gas to flow from the second fluid source 38. This fluid is then directed toward the fluid analyzer device 28 to calibrate the fluid analyzer device 28 by providing the fluid having a known quantity of contaminant (i.e., a known concentration of carbon dioxide) through the fluid analyzer device 28. The output value of the fluid analyzer device 28 is then electronically adjusted to correspond mathematically to the known concentration of contaminant (e.g., carbon dioxide) in the fluid flow path 24.

With particular reference to FIG. 2, the first mode of operation begins at step 100 by providing zero gas to the fluid flow path 24 from the first fluid source 36 by opening a valve 37 associated with the first fluid source 36. In step 102, the contaminant (e.g., carbon dioxide) concentration of the zero gas is determined by the fluid analyzer device 28. In step 103, the output value of the fluid analyzer device 28 is adjusted electronically to correspond to a zero value. In step 104, the flow of zero gas is terminated by closing valve 37. In step 106, span gas is provided to the fluid flow path 24 from the second fluid source 38 by opening valve 39. In step 108, the contaminant (i.e., carbon dioxide) concentration of the span gas is determined by the fluid analyzer device 28.

In step 109, the output value of the fluid analyzer device 28 is adjusted electronically to correspond to the mathematical equivalent on the output scale of the concentration of contaminant (i.e., carbon monoxide) in the span gas supplied. In step 110, the flow of span gas through the fluid flow path 24 is terminated by closing valve 39. A calibration timer is started at 111. In step 112, steps 100 through 104 are repeated to more precisely align the output value at step 103 with the concentration of contaminant in the zero gas. In step 114, the calibration timer is terminated when a predetermined amount of time has elapsed, and the first mode of operation is restarted at step 100.

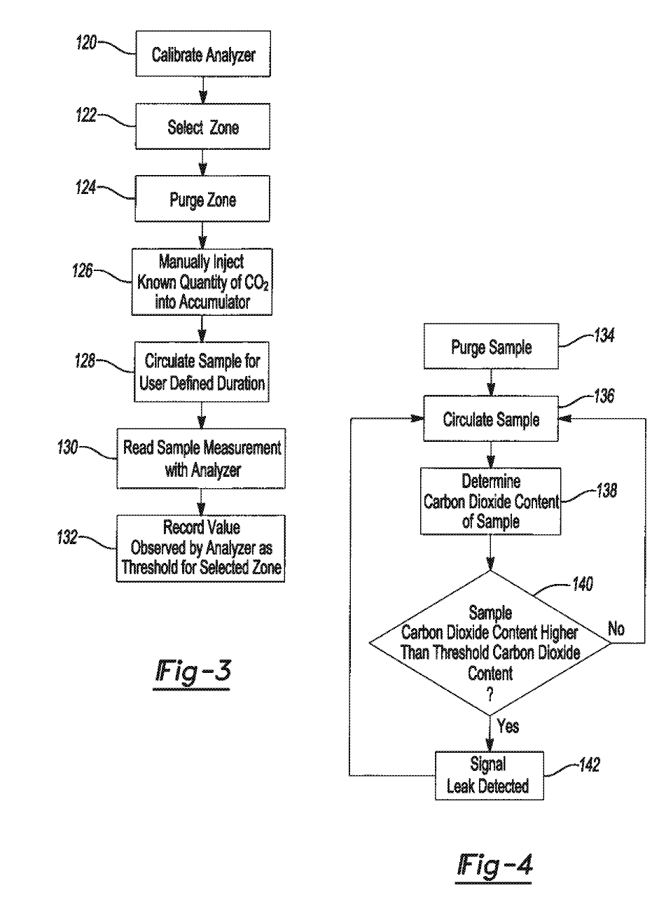

With reference to FIG. 3 in a second mode of operation, the system 10 determines a threshold output value of the fluid analyzer device 28 for a known quantity of carbon dioxide that may be indicative of a head-gasket failure. The second mode of operation begins at step 120 by calibrating the fluid analyzer device 28 in the manner previously described in steps 100 through 114 (FIG. 2). At step 122, the system 10 selects the zone or engine 12 when more than one engine 12 is operatively associated with the system 10. At step 124, the system 10 purges the fluid flow path 24 using zero gas from the first fluid source 36 by directing the zero gas through the fluid flow path 24, the circulation flow path 24a, and the fluid analyzer device 28, before expelling the zero gas from the system 10 at first exhaust valve 35a by opening valves 51, 53, 40 and 35a.

At step 126, valves 51, 53, 40 and 35a are closed and a known quantity of carbon dioxide is injected or otherwise added to the accumulator 16. The known quantity of carbon dioxide is a quantity that is indicative of a head-gasket failure. At step 128, the known quantity of carbon dioxide is circulated through the system 10 and the fluid flow path 24. At step 130, the fluid analyzer device 28 determines the threshold output value associated with the known quantity of carbon dioxide. At step 132, the threshold output value for the engine 12 is recorded.

With reference to FIGS. 1 and 4, in a third mode of operation, the system 10 analyzes a fluid sample from the accumulator 16 using the fluid analyzer device 28. During a first stage of the third mode of operation, the first fluid source 36 is placed in fluid communication with the fluid flow path 24 to provide zero gas to at least a portion of the fluid flow path 24. Namely, valves 37 and 40 are opened such that zero gas from the first fluid source 36 flows into the input port 20 of the accumulator 16. As the zero gas flows into the accumulator 16, the zero gas forces other fluid located within the accumulator 16 (e.g., a sample gas) through the outlet port 22 of the accumulator 16. The first exhaust valve 35a is opened such that the flow of zero gas from the first fluid source 36 forces the zero gas and other fluids through fluid flow path 24 and out of the first exhaust valve 35a. In so doing, the zero gas essentially purges the system 10.

During a second stage of the third mode of operation, the second valve 42 is closed such that the fluid flow path 24 is in fluid communication with the circulation flow path 24a to circulate a sample fluid (e.g., air) through the system 10. The sample fluid flows through the accumulator 16, the outlet port 22, the filter 30, and the heated gas transfer conduit 32 prior to reaching the circulation flow path 24a. During the second stage, power is supplied to the pump 34 such that the pump 34 circulates the sample fluid through the circulation flow path 24a and the fluid flow path 24. This second stage allows the system 10 to accumulate a sufficient sample gas within the accumulator 16 during operation of the engine 12.

During a third stage of the third mode of operation, the second valve 42 is opened such that the pump 34 causes the sample fluid to flow from the accumulator 16, through the fluid analyzer device 28, and back to the accumulator 16.

With particular reference to FIG. 4, the third mode of operation begins at step 134 by purging sample gas from the fluid flow path 24 with zero gas from the first fluid source 36 in the manner previously described. Namely, the valves 37, 46 are opened to cause zero gas to flow through the fluid analyzer device 28 prior to being expelled at the second exhaust valve 35b. At step 136, sample gas from the accumulator 16 is circulated through the circulation flow path 24a and/or through the fluid analyzer device 28 for a predetermined length of time using the pump 34. At step 138, the fluid analyzer device 28 determines the output value of the carbon dioxide content in the sample gas. At step 140, the fluid analyzer device 28 compares the output value of the carbon dioxide content in the sample gas with the threshold output value for a known quantity of carbon dioxide (FIG. 3). The threshold output value for carbon dioxide content may be a value indicative of a head-gasket failure, or other failure in the sealed interface between the cylinder(s) and the cooling system 14, as described above with respect to FIG. 3.

If the output value of the carbon dioxide content in the sample gas is greater than the threshold output value, the system 10 signals that a head-gasket failure has been detected (step 142). If the output value of the carbon dioxide content in the sample gas is less than the threshold output value, the system 10 proceeds to step 136 and circulates a second sample gas (for example, from a second engine) through the circulation flow path 24a. Determination of the content of carbon dioxide in the sample gas is performed by the fluid analyzer device 28.

Comparison of the output value of the carbon dioxide content in the sample gas with the threshold output value can be performed by a processor 100 (FIG. 1) associated with or remotely located from the fluid analyzer device 28. If the processor 100 is remotely located from the fluid analyzer device 28 the processor 100 is in communication with the fluid analyzer device 28 via wired and/or wireless communication to allow the fluid analyzer device 28 to communicate measured output values (i.e., carbon dioxide content) to the processor 100 for comparison to the threshold output value. The processor 100 then determines whether the determined carbon dioxide content exceeds the threshold output value and, if so, whether the engine 12 has experienced a head-gasket failure.

As described, the system 10 is used in conjunction with an engine 12 to determine whether a head gasket properly seals an interface between a cylinder and a cylinder head. Namely, the system 10 selectively measures sample gas from within the accumulator 16 of the cooling system 14 to determine whether a predetermined amount of carbon dioxide is present within the sample gas.

The system 10 may be used in conjunction with an engine 12 or a series of engines 12 respectively connected to a test fixture such as an engine dynamometer (not shown). The system 10 monitors the engine 12 or engines 12 in real time while the engines 12 are running in the dynamometer. The following process is used while the engine(s) 12 are running and, as a result, is used in real time without requiring the engine(s) 12 to be stopped or partially disassembled.

The system 10 first calibrates the fluid analyzer device 28 by following the procedure set forth at FIG. 2. Namely, the system 10 purges the fluid analyzer device 28 by directing a stream of zero gas from the first fluid source 36 through the fluid analyzer device 28. The zero gas is then expelled downstream of the fluid analyzer device 28 at the second exhaust valve 35b.

Once the fluid analyzer device 28 is output adjusted for zero, valves 37, 46 are closed and the valve 39 associated with the second fluid source 38 is opened. Span gas from the second fluid source 38 is directed toward the fluid analyzer device 28 to calibrate the fluid analyzer device 28. Following calibration, zero gas is once again directed through the fluid analyzer device 28 and is expelled at the second exhaust valve 35b to fine tune the zero calibration of the fluid analyzer device 28.

Following calibration, the system 10 may be injected with a known quantity of carbon dioxide in an effort to set a threshold output value for an engine 12 under test. Namely, a known quantity of carbon dioxide may be injected into the accumulator 16 for the particular engine 12 under test. The gas injected into the accumulator 16 is drawn into the circulation flow path 24a by the pump 34 to direct the sample from the accumulator 16 through the fluid analyzer device 28. The gas is circulated for a predetermined duration and is analyzed by the fluid analyzer device 28. The value observed by the fluid analyzer device 28 is recorded by the system 10 as a threshold output value for the particular engine 12. This threshold output value is then used by the processor 100 for comparison to real-time samples taken during operation of the engine 12.

Prior to measuring a sample of gas from the accumulator 16 during operation of an engine 12, the system 10 first purges the fluid analyzer device 28 with zero gas from the first fluid source 36. Once the fluid analyzer device 28 is sufficiently purged, the remaining zero gas and the fluid disposed within the accumulator 16 mix to form a sample gas, which is directed to the circulation flow path 24a by the pump 34.

The sample gas passes through the fluid analyzer device 28 to determine whether a carbon dioxide content within the sample gas is higher than the threshold carbon dioxide content for the particular engine 12 as set forth at step 140 of FIG. 4. If the carbon dioxide content of the sample gas is higher than the threshold carbon dioxide content, a leak signal is declared by the system 10 at step 142 of FIG. 4. If the carbon dioxide content of the sample gas is lower than the threshold carbon dioxide content, a leak signal is not declared and the system 10 continues to operate during operation of the engine 12.

The system 10 may circulate a sample gas through the fluid analyzer device 28 periodically during operation of the engine 12. Namely, the system 10 may direct zero gas from the first fluid source 36 to the accumulator 16 at predetermined intervals and circulate the same for a predetermined duration to allow the fluid analyzer device 28 to compare a carbon dioxide content of the sample gas to the threshold carbon dioxide content at predetermined intervals. Regardless of the frequency with which the system 10 directs sample gas through the fluid analyzer device 28, the system 10 detects whether the engine(s) 12 experiences a head-gasket failure while the engine(s) 12 is running.

While the foregoing example describes operation of the system 10 in conjunction with an engine 12 or engines 12 associated with a test fixture, the system 10 could be used in conjunction with an engine 12 that is installed in a vehicle (not shown). For example, the system 10 may be selectively connected to the accumulator 16 of the vehicle at ports of a cap (none shown) of the accumulator 16. In this way, the system 10 may be used in conjunction with the vehicle to determine whether the engine 12 is experiencing a head-gasket failure without disassembly of the engine 12 or removal of the engine 12 from the vehicle.

Once the system 10 is properly attached to the accumulator 16, the foregoing methodologies set forth at FIGS. 2-4 can be followed to determine whether the engine 12 is experiencing a head-gasket failure. As with an engine 12 disposed within a test fixture, the engine 12 associated with the vehicle does not need to be removed from the vehicle to determine whether the engine 12 is experiencing a head-gasket failure and, further, is determined when the engine 12 is running in the vehicle.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for determining the existence of a head-gasket failure in an engine, comprising:
providing an engine and an engine cooling system in fluid communication with the engine;
providing an accumulator in fluid communication with the engine cooling system, the accumulator including a quantity of sample gas;
providing a flow path in fluid communication with the accumulator, the flow path including at least one gas analyzer;
circulating at least a portion of the sample gas through the flow path including the at least one gas analyzer; and
determining an amount of carbon dioxide in the sample gas.

2. The method of claim 1, further comprising declaring a head-gasket failure if the amount of carbon dioxide in the sample gas exceeds a predetermined amount.

3. The method of claim 1, further comprising circulating a zero gas through the flow path to purge the at least one gas analyzer prior to circulating the sample gas through the at least one analyzer.

4. The method of claim 1, wherein circulating the sample gas through the flow path includes circulating the sample gas through the flow path when the engine is running.

5. The method of claim 1, further comprising supplying zero gas to the accumulator to direct the sample gas from the accumulator to the at least one analyzer.

6. The method of claim 5, wherein directing the sample gas to the at least one analyzer includes imparting a fluid force on the sample gas via a pump.

7. The method of claim 1, further comprising calibrating the at least one analyzer with a span gas having a known quantity of carbon dioxide.

8. The method of claim 7, wherein calibrating the at least one analyzer includes purging the at least one analyzer with zero gas prior to circulating the span gas through the at least one analyzer.

9. The method of claim 7, wherein calibrating the at least one analyzer includes purging the at least one analyzer with zero gas after circulating the span gas through the at least one analyzer.

10. The method of claim 1, further comprising adding a predetermined quantity of carbon dioxide to the accumulator, circulating the predetermined quantity of carbon dioxide through the flow path including the at least one gas analyzer, and determining a threshold value for the known quantity of carbon dioxide.

11. The method of claim 10, further comprising comparing the threshold value to the amount of carbon dioxide in the sample gas.

12. The method of claim 11, further comprising declaring a head-gasket failure if the amount of carbon dioxide in the sample gas exceeds the threshold value.

13. An apparatus for determining the existence of a head-gasket failure in an engine, the apparatus comprising:
an accumulator in fluid communication with the engine;
an engine coolant flow path in fluid communication with the engine and with the accumulator;
a gas flow path fluidly coupled to the accumulator; and
at least one gas analyzer fluidly coupled to the accumulator via the gas flow path and operable to receive a sample gas from the accumulator via the gas flow path, the at least one gas analyzer operable to detect an amount of carbon dioxide in the sample gas.

14. The apparatus of claim 13, wherein the at least one gas analyzer determines a head-gasket failure when the amount of carbon dioxide in the sample gas exceeds a predetermined amount.

15. The apparatus of claim 13, further comprising a pump operable to direct the sample gas from the accumulator through the at least one analyzer.

16. The apparatus of claim 13, further comprising a source of zero gas and a source of span gas each in fluid communication with the gas flow path.

17. The apparatus of claim 16, wherein the zero gas is selectively supplied to the at least one analyzer to purge the at least one analyzer.

18. The apparatus of claim 16, wherein the span gas is selectively supplied to the at least one analyzer to calibrate the at least one analyzer.

19. The apparatus of claim 13, wherein the accumulator includes an inlet and an outlet, said inlet selectively receiving one of a zero gas and a span gas.

20. The apparatus of claim 19, wherein the inlet receives zero gas to displace the sample gas from the outlet to allow the sample gas to enter the gas flow path and be analyzed by the at least one analyzer.
